(19) United States
(12) Patent Application Publication
(10) Pub. No.: US 2016/0206658 A1
(43) Pub. Date: Jul. 21, 2016

(54) NUTRITION FOR PREVENTION OF INFECTIONS

(71) Applicant: N.V. Nutricia, Zoetermeer (NL)

(72) Inventors: Jan KNOL, Wageningen (NL); Cornelus Johannes Petrus VAN LIMPT, Amersfoort (NL); Francis LE CROIX, Godewaersvelde (FR); Valérie PETAY, Nieuwkerke-Heuvelland (BE)

(73) Assignee: N.V. Nutricia, Zoetermeer (NL)

(21) Appl. No.: 14/952,440

(22) Filed: Nov. 25, 2015

Related U.S. Application Data
(63) Continuation of application No. 14/201,536, filed on Mar. 7, 2014, now abandoned.
Application No. 14/201,536 is a continuation of application No. 12/997,541, filed on Dec. 10, 2010, now abandoned.

(30) Foreign Application Priority Data
Jun. 13, 2008 (NL) PCT/NL08/50375
Oct. 31, 2008 (EP) 08168054.8

(51) Int. Cl. A61K 35/20; A61K 31/733; A61K 31/702

(52) U.S. Cl. CPC A61K 35/20; A61K 31/702; A61K 31/733; A23L 1/296; A23L 1/3014; A23L 1/308; A23Y 2240/75; A23Y 2300/29; A23V 2002/00

(57) ABSTRACT

A nutritional composition comprising a combination of non-digestible oligosaccharides and a product obtained by incubating an aqueous substrate by bifidobacteria and optionally a product obtained by incubating an aqueous substrate by S. thermophilus. Said combination reduces bacterial translocation and improves the intestinal barrier function.

### CROSS-REFERENCE TO RELATED APPLICATIONS

This application is a Continuation of U.S. patent application Ser. No. 14/201,536, filed Mar. 7, 2014, as a Continuation of U.S. patent application Ser. No. 12/997,541, filed as the National Phase Application of International Patent Application No. PCT/NL2009/050330, filed Jun. 12, 2009, published as WO 2009/151329, which claims priority to European Application No. 08168054.8, filed Oct. 31, 2008 and International Application No. PCT/NL2008/050375, filed Jun. 13, 2008. The contents of these applications are herein incorporated by reference in their entirety.

### FIELD OF THE INVENTION

The present invention relates to a nutritional composition, in particular an infant nutrition, for decreasing intestinal barrier permeability and/or treating or prevention of infections.

### BACKGROUND OF THE INVENTION

The barrier of the intestine is immature in the newborn infant. As a result of a decreased intestinal barrier, or increased intestinal permeability, newborn infants are prone to bacterial translocation and translocation of toxins and allergens. Infants fed a conventional infant formula have an even increased intestinal permeability function compared to breast fed infants.

One solution is to use probiotic bacteria to treat disorders associated with increased intestinal permeability. However, the use of probiotics, being living micro-organisms themselves, in subjects with a decreased intestinal barrier function may not be safe as in some cases these probiotics are able translocate across the intestinal barrier and cause systemic infections themselves. Another solution is to use prebiotics.

EP 1815755 discloses a nutritional composition comprising long chain poly-unsaturated fatty acids and two distinct non-digestible oligosaccharides for improvement of barrier function.

EP 1320375 discloses the use of dietary fibre, particularly fructan, for the manufacture of a composition for inhibiting the systemic growth of pathogenic bacteria. WO 2004/112509 pertains to a composition for inducing a pattern of gut barrier maturation similar to that observed with breast-feeding and able to improve gut barrier maturation, containing a combination of specific ingredients designed to provide a synergistic effect all along gastrointestinal tract and barrier function.

WO 2005/039319 relates to a preparation comprising Bifidobacterium breve and a mixture of non-digestible carbohydrates for non- or partially breast-fed infants as well as the use thereof for the treatment or prevention of immune disorders in non- or partially breast-fed infants.

WO 2007/067053 discloses infant formula comprising the plant-derived prebiotics inulin and galacturonic acid oligosaccharide and the from lactose synthesized prebiotic transgalacto-oligosaccharide to reduce infections.

WO 2004/069156 discloses the use of inactivated bacteria.

Heyman et al, 2005, Acta Paediatr. 94:34-36 disclose the effects of a milk formula fermented with Bifidobacterium breve and Streptococcus thermophilus and heated/dehydrated to inactivate the micro-organisms to decrease the intestinal permeability to macromolecules and to reinforce the intestinal barrier resistance to food proteins.

There is however a need for further development of infant formula for improving intestinal barrier function and reduction of infections.

### SUMMARY OF THE INVENTION

The present inventors have surprisingly found that a combination of i) a product obtained by incubating an aqueous substrate with bifidobacteria, wherein the substrate is at least one selected from the group consisting of milk, milk protein, whey, whey protein, whey protein hydrolysate, casein hydrolysate, and lactose and subsequently inactivating the bifidobacteria by heating the incubated mixture and/or removing the bifidobacteria cells from the incubated mixture by centrifugation and/or filtration and ii) at least two different non-digestible carbohydrates, wherein at least one, preferably two, is selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, raffinose, lactosucrose, sialic acid comprising oligosaccharides and uronic acid oligosaccharides, synergistically increase resistance against infections. A decreased bacterial translocation across the intestinal barrier was observed in animals having consumed the present composition, compared to animals having consumed the single components. This is indicative of an increased intestinal barrier function or decreased intestinal barrier permeability.

The synergistic effect between the combination of the ingredients i) and ii) as defined above is surprising. It cannot be explained by a symbiotic effect, wherein the non-digestible carbohydrates (ii)) are specifically stimulating the growth of the beneficial micro-organisms present in the same preparation, since no living cells of bifidobacteria are present in the present preparation.

Removal and/or inactivation of living bifidobacteria cells has the further advantage that the composition can be pasteurised and/or sterilised, consequently reducing the chance of contamination with harmful micro-organisms. This is especially advantageous in infants, since infants have an increased intestinal permeability. Additionally, since the bifidobacteria bacteria are removed or inactivated they cannot cause infections themselves.

A further advantage is that the dose of bioactive components received by each human subject can be better controlled. Also advantageously storage of the product is more easily and with reduced costs. Furthermore, advantageously no post-acidification occurs in stored products, thereby avoiding adverse effect relating to coagulation of proteins and adverse taste. Still a further advantage is that inactivated and/or removed bifidobacteria no longer able to breakdown and consume the non-digestible carbohydrates.

The present preparation is suitable for treatment and/or prevention of infections, especially systemic infections; for prevention and/or treatment of diarrhoea; for reduction of bacterial translocation, preferably reduction of bacterial translocation across the intestinal barrier; and/or for improving intestinal barrier function.

### DETAILED DESCRIPTION OF THE INVENTION

The present invention concerns a process for the manufacture of a preparation comprising the steps of:
a: incubating an aqueous substrate with bifidobacteria, wherein the substrate comprises at least one selected from the group consisting of milk, milk protein, whey, whey protein, whey protein hydrolysate, casein hydrolysate, and lactose to obtain an incubated mixture;
b: inactivating the bifidobacteria by heating the incubated mixture and/or removing bifidobacteria cells from the incubated mixture by centrifugation and/or filtration; and
c: combining a composition comprising the mixture obtained in step a or obtained in step b, preferably obtained in step b with at least two different non-digestible carbohydrates, wherein at least one, preferably two, is selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, raffinose, lactosucrose, sialic acid comprising oligosaccharides and uronic acid oligosaccharides.

In one aspect, the present invention concerns a preparation obtainable by the process according to the present invention. In one embodiment the invention concerns a nutritional composition comprising or consisting of the preparation obtainable by the process according to the present invention.

Also the invention concerns a method for the treatment and/or prevention of a disease in a mammal, said method comprising administering the present preparation to the mammal

Also the invention concerns a method for providing nutrition to an infant, said method comprising administering the present preparation or nutritional composition comprising the present preparation to the infant.

Process Comprising Incubating a Substrate with Bifidobacteria

The present invention concerns a preparation obtainable or obtained by incubating an aqueous substrate with bifidobacteria, wherein the aqueous substrate comprises at least one selected from the group consisting of milk, milk protein, whey, whey protein, whey protein hydrolysate, casein hydrolysate, and lactose (hereinafter referred to as step (a)). The incubated mixture obtained or obtainable in step (a) is subjected to a step (b) comprising an inactivation step by heat treatment and/or removal step of the bifidobacteria cells by centrifugation and/or filtration. Step (b) is performed in order to reduce the amount of living bifidobacteria in the preparation, preferably by at least 90%, more preferably by at least 99%. In one embodiment the incubation step comprises a fermentation step and/or bioconversion step. During fermentation the aqueous substrate is fermented by the bifidobacteria. During bioconversion the aqueous substrate is bioconversed by the bifidobacteria.

The preparation obtained or obtainable by (hereafter wherever only ‘obtained’ is mentioned, also ‘obtainable by’ is meant) the present process preferably comprises bacterial cell fragments like glycoproteins, glycolipids, peptidoglycan, lipoteichoic acid (LTA), lipoproteins, DNA, and/or capsular polysaccharides. These fragments evoke an immunological response. It is of advantage to use the product obtained by incubating an aqueous substrate comprising at least one selected from the group consisting of milk, milk protein, whey, whey protein, whey protein hydrolysate, casein hydrolysate and lactose with bifidobacteria and subsequently inactivating and/or removing the bifidobacteria, since this will result in a higher concentration of bacterial cell fragments. Upon incubation of the aqueous substrate with the bifidobacteria, additional bio-active compounds may be formed, such as organic acids, bioactive peptides and/or oligosaccharides, which stimulate the immune system. When commercial preparations of probiotics are used, the probiotic bacterial cells are usually washed and separated from the aqueous growth medium that comprised the bacterial cell fragments, thereby strongly reducing or even eliminating the supernatant of the incubates substrate comprising the bacterial cell fragments. In the present invention this is not the case. The presence of intact cells (living or dead) is not necessary for the immune response; the aqueous substrate itself, after the present incubation step with bifidobacteria, has already beneficial effects on the immune system.

### Bifidobacteria and Streptococci

Bifidobacteria used for the present process are preferably provided as a mono- or mixed culture. Bifidobacteria are Gram-positive, anaerobic, rod-shaped lactic acid producing bacteria. The present Bifidobacterium species preferably have at least 95% identity of the 16 S rRNA sequence when compared to the type strain of the respective Bifidobacterium species, more preferably at least 97% identity as defined in handbooks on this subject for instance Sambrook, J., Fritsch, E. F., and Maniatis, T. (1989), Molecular Cloning, A Laboratory Manual, 2nd ed., Cold Spring Harbor (N.Y.) Laboratory Press. The Bifodobacteria preferably used are also described by Scardovi, V. Genus Bifidobacterium. p.1418 - p.1434. In: Bergey's manual of systematic Bacteriology. Vol. 2. Sneath, P.H.A., N. S. Mair, M. E. Sharpe and J. G. Holt (ed.). Baltimore: Williams & Wilkins. 1986. 635 p.

Preferably the bifidobacteria used in producing the present preparation is at least one Bifidobacterium selected from the group consisting of B. breve, B. infantis, B. bifidum, B. catenulatum, B. adolescentis, B. thermophilum, B. gallicum, B. animalis or lactis, B. angulatum, B. pseudocatenulatum, B. thermacidophilum and B. longum more preferably B. breve, B. infantis, B. bifidum, B. catenulatum, B. longum, more preferably B. longum and B. breve, even more preferably B. breve, most preferably B. breve 1-2219 deposited at the Collection Nationale de Cultures de Microorganisms van Institute Pasteur, Paris, France on May 31, 1999 by Compagnie Gervais Danone. This strain was published in WO 2004/093899.

Preferably the composition also comprises a product obtained by incubating an aqueous substrate comprising at least one selected from the group consisting of milk, milk protein, whey, whey protein, whey protein hydrolysate, casein, casein hydrolysate and lactose, preferably the group of whey and lactose with streptococci and preferably subsequently inactivating and/or removing the streptococci. Streptococci are Gram-positive, anaerobic, coccoid-shaped lactic acid producing bacteria. The Streptococcus species preferably have at least 95% identity of the 16 S rRNA sequence when compared to the type strain of the respective Streptococcus species, more preferably at least 97% identity as defined in handbooks on this subject for instance Sambrook, J., Fritsch, E. F., and Maniatis, T. (1989), Molecular Cloning, A Laboratory Manual, 2nd ed., Cold Spring Harbor (N.Y.) Laboratory Press. Preferably production of a product obtained by incubating an aqueous substrate with streptococci and subsequently inactivating the streptococci is performed with Streptococcus species selected from the group consisting of S. salivarius and S. thermophilus, more preferably S. thermophilus, even more preferably strain S. thermophilus CNCM 1-1620 or strain CNCM 1-1470, most preferably strain CNCN 1-1620. S. thermophilus CNCM 1-1620 and 1-1470 advantageously produces high amounts of beta-galactosidase. S. thermophilus CNCM 1-1620 was deposited under the Budapest Treaty on Aug. 23, 1995 at Collection Nationale de Cultures de Microorganisms van Institute Pasteur, Paris, France by Compagnie Gervais Danone. S. thermophilus CNCM 1-1470 was deposited under the Budapest Treaty on Aug. 25, 1994 at Collection Nationale de Cultures de Microorganisms van Institute Pasteur, Paris, France by Compagnie Gervais Danone. These strains were published in EP 778885.

### Process Step a) Incubation the Aqueous Substrate

### Step (a) is Preferably Performed By:

a1 inoculating bifidobacteria in the aqueous substrate in amount of between 1×10² to 1×10¹¹ cfu bifidobacteria/ml, said aqueous substrate having a pH of between 4 and 8, and comprising at least one selected from the group consisting of milk, whey, whey protein, whey protein hydrolysate, casein hydrolysate, and lactose,
a2 incubating said bifidobacteria in said aqueous medium, under aerobic or anaerobic conditions and at a temperature of 20° C. to 50° C., for at least 2 h.

The aqueous substrate to be incubated with bifidobacteria comprises at least one, more preferably at least two, selected from the group consisting of milk, whey, whey protein, whey protein hydrolysate, casein hydrolysate, and lactose. Preferably the substrate does not comprise intact casein. It was found that less immunostimulatory substances were formed when the aqueous substrate comprised high amounts of intact casein. Therefore the aqueous substrate comprises preferably less than 25 g/l casein, more preferably less than 15 g/l, even more preferably less than 5g/l, most preferably less than 1 g/l intact casein. The aqueous substrate therefore even more preferably comprises whey and/or whey protein and/or whey protein hydrolysate.

Milk can be whole milk, semi-skimmed milk and/or skimmed milk. Preferably skimmed milk is used. Whey can be sweet whey, acid whey or whey from which the casein has been removed for example by filtration or whey permeate. Preferably the whey is present in a concentration of 3 to 80 g dry weight per liter (l) aqueous substrate, more preferably 40 to 60 g per l. Preferably whey protein concentrate is used. Preferably whey protein hydrolysate is used and is present in an amount of 2 to 80 g dry weight per l aqueous substrate, more preferably 5 to15 g/l. Preferably lactose is present in an amount of 5 to 50 g dry weight per l aqueous substrate, more preferably 1 to 30 g/l. Preferably the aqueous substrate comprises buffer salts in order to keep the pH within a desired range. Preferably sodium or potassium dihydrogen phosphate is used as buffer salt, preferably in an amount of 0.5 to 5 g/l, more preferably 1.5 to 3 g per l. Preferably the aqueous substrate comprises cysteine in amount of 0.1 to 0.5 g per l aqueous substrate, more preferably 0.2 to 0.4 g/l. The presence of cysteine results in low redox potential of the substrate which is advantageous for activity of lactic acid producing bacteria, particularly bifidobacteria. Preferably the aqueous substrate comprises yeast extract in an amount of 0.5 to 5 g/l aqueous substrate, more preferably 1.5 to 3 g/l. Yeast extract is a rich source of enzyme co-factors and growth factors for lactic acid producing bacteria. The presence of yeast extract will enhance the bioconversion and/or fermentation by bifidobacteria.

Preferably the aqueous substrate to be incubated comprises a high concentration of solids, preferably more than 20 wt. % solids based on volume, more preferably more than 40 wt. % solids. A high concentration is advantageous when performing the further processing steps, such as for example spray drying, centrifugation or filtration.

Suitably the aqueous substrate is pasteurised before the incubation step, in order to eliminate the presence of unwanted living bacteria. Suitably the product is pasteurised after incubation, in order to inactivate enzymes. Suitably the enzyme inactivation takes place at 75° C. for 1 min. Suitably the enzyme inactivation takes place at 75° C. for 3 min. Suitably the aqueous substrate is homogenised before and/or after the incubation step. Homogenisation results in a more stable product, especially in the presence of fat (lipids).

The inoculation density is preferably between 1×10² to 1×10¹¹, preferably between 1×10⁴ to 1×10¹° cfu bifidobacteria per ml aqueous substrate, more preferably between 1×10² to 1×10⁹ cfu bifidobacteria/ml aqueous substrate. Methods for obtaining a concentrated starter culture of bifidobacteria to be inoculated in the aqueous substrate are known in the art. The final bacteria density of bifidobacteria after incubation is preferably between 1×10³ to 1×10¹¹ more preferably between 1×10⁴ to 1×10⁹ cfu/ml aqueous substrate.

The incubation with bifidobacteria is preferably performed at a temperature of approximately 20° C. to 50° C., more preferably 30° C. to 45° C., even more preferably approximately 37° C. to 42° C. The optimum temperature for growth and/or activity for bifidobacteria is between 37° C. and 42° C.

The incubation with bifidobacteria is preferably under anaerobic conditions, since the growth of bifidobacteria and the enzymatic activity of many enzymes of bifidobacteria are impaired under aerobic conditions. However acidification is not always desired. Thus, in one embodiment, the incubation step suitably takes place under aerobic conditions.

The incubation with bifidobacteria is preferably performed at a pH of 4 to 8, more preferably 5.6 to 7.5, even more preferably 6 to 7.5. This pH does not induce protein precipitation and/or an adverse taste, while at the same time bifidobacteria are able to interact with the aqueous substrate.

The incubation time is preferably at least 2 h, preferably between 4 and 48 h, more preferably between 6 and 24 h, even more preferably between 6 and 15 h. A sufficient long time enables the interaction between the bifidobacteria and the aqueous substrate and/or the production of cell fragments such as glycoproteins, glycolipids, peptidoglycan, lipoteichoic acid (LTA), lipoproteins, DNA and/or capsular polysaccharides to take place to a large extent, whereas the incubation time need not be unnecessarily long for economical reasons.

Methods of Inactivation and/or Physically Removal of Living Cells of Bifidobacteria

In step (b) of the present process living cells bifidobacteria are after incubation in step a) preferably essentially all eliminated, for example by inactivation by heat treatment and/or physical removal. The cells are preferably inactivated by heat treatment. Preferably the bifidobacteria are heat killed after incubation step a). Preferable ways of heat killing are pasteurization, sterilization, ultra high temperature treatment, spray cooking and/or spray drying at temperatures bifidobacteria do not survive. The heat treatment is preferably performed at at least 50° C., more preferably at at least 65° C. The heat treatment is preferably performed for at least 5 minutes, more preferably for at least 10 minutes. The heat treatment is preferably performed for at least 5 minutes at at least 50° C., more preferably for at least 10 minutes at at least 65° C. The heat treatment is preferably performed for at least 1 minutes at at least 75° C., more preferably for at least 3 minutes at at least 75° C. Preferably intact cells of bifidobacteria are removed from the incubated product by physical elimination such as filtration and/or centrifugation, for example centrifugation for 1 h at 3000 g, with the intact cells remaining in the pellet or retentate and the product obtained by incubating a milk and/or milk-derived substrate with bifidobacteria and subsequently inactivating the bifidobacteria cell fragments remaining in the supernatant and/or filtrate, respectively.

The heat inactivation and/or physical removal of living cells is such that the amount of living bifidobacteria after treatment is below the detection limit as used by conventional plating techniques known in the art. This detection limit is less than 10⁴ cfu living cells of bifidobacteria based on g dry weight composition, more preferably less than 10³ cfu/g. Hence, preferably in one embodiment according to the invention the preparation after step b comprises less than 10³ cfu living bifidobacteria per g dry weight of the preparation Preferably the heat inactivation and/or removal step is such that at least 90, more preferably at least 99% of the cells present in the incubated mixture after step a) is eliminated.

The requirement that living cells are inactivated has the advantage that, after production, the final nutritional composition can be pasteurised and/or sterilised, consequently reducing the chance of contamination with harmful micro-organisms. So the present invention enables liquid, ready-to-use formula to be prepared and stored at room temperature. Furthermore, the dose of bioactive components received by each human subject can be more easily controlled, since no further growth in a liquid product occurs, nor growth in the intestinal tract of the human subject. The latter is a variable factor depending on the individual's intestinal environment, and thereby leads to variations in the extent of beneficial effects in individual subjects. Still a further advantage is that inactivated and/or removed bifidobacteria and streptococci no longer are able to breakdown and consume the non-digestible carbohydrates.

Additional advantages are that the nutritional composition can be stored more easily and with reduced costs, since no special precautions have to be taken to maintain the viability of bifidobacteria at an acceptable level. This is especially the case in products with a water activity above 0.3. Also no post-acidification occurs in stored products with a high water activity and/or in powdered nutritional compositions in the period after reconstitution with water and before consumption. Adverse effects relating to coagulation of proteins and adverse taste are avoided in this way.

### Addition of Additional Components and Other Optional Process Steps

Optionally one or more of the following steps may follow the above process step b):
i) Ultrafiltrating the product after incubation through filtration membranes having a cut-off threshold between 100 and 300 kDa, so as to obtain a concentrated retentate. The membranes are preferably polyethersulfone membranes and filtration is preferably performed at a temperature below 60° C.
ii) Washing the concentrated retentate with water.
iii) Dehydrating the concentrated retentate, preferably by lyophilisation.
iv) Dissolving the dehydrated retentate in a buffer, preferably a Tris buffer with pH 6-8.
v) Performing gel exclusion chromatography of the retentate solution, on a column having an exclusion threshold of 600 kDa, preferably a Dextran or agarose column such as Superdex ®200.
vi) Recovery of the filtered or excluded fraction at the end of the chromatography.
vii) Desalting the product with a membrane with a cut-off of 10 kDa. Recovering the excluded fraction at the end of the chromatography.

These steps are preferably performed under sterile conditions. Additional ingredients that may be beneficial for obtaining the desired final nutritional composition may be added after process step a), preferably immediately prior to step b) or after process step b). Preferably these are added after step b). For an infant milk formula, ingredients such as skimmed milk, whey, lactose, vegetable fat, minerals, vitamins, as known in the art may be added.

Preferably, an aqueous substrate comprising whey, whey protein and/or whey protein hydrolysate, is pasteurized, cooled and incubated with one or more Bifidobacterium strains, preferably B. breve strain CNCM 1-2219, upon which the incubated product is heat treated and stored. Optionally the incubated product is mixed with other components making up the nutritional composition. A fat component may or may not be included, but preferably a fat component is not yet included at this stage. Preferably, the mixture is preheated, and subsequently fat (also the term ‘lipids’ is used herein) is added in-line, homogenized, heat-treated and dried.

Another preferred method for preparing the incubated product of the present invention is disclosed in WO 01/01785, more particular in example 1 and 2. Another preferred method for preparing the incubated product of the present invention is described in WO 2004/093899, more particularly in example 1.

Additional ingredients that may be beneficial for obtaining the desired final nutritional composition may be added after process step a) or b). Preferably these are added after step b). For an infant milk formula, ingredients such as skimmed milk, whey, lactose, vegetable fat, minerals, vitamins, as known in the art may be added.

Preferably the final nutritional composition comprises from 5 to 100 wt. % based on dry weight of the preparation obtained by step b, more preferably from 5 to 99.5 wt. %, more preferably from 5 to 95 wt. %, even more preferably from 5 to 80 wt. %, even more preferably from 5 to 40 wt. %, most preferably from 10 to 40 wt. %. Preferably, the final nutritional composition comprises from 0.5 to 20 wt. % of a product obtained by step b per 100 ml, more preferably 0.5 to 14 wt. %, more preferably 1 to 10 wt. %, even more preferably 1 to 5 wt. % per 100 ml.

Preferably the present final nutritional composition comprises inactivated bifidobacteria and/or bacterial fragments derived from bifidobacteria obtained from more than 1×10³ cfu bifidobacteria per g, based on dry weight of the final composition, more preferably more than 1×10⁴cfu, even more preferably more than 1×10⁵ cfu. Preferably the inactivated bifidobacteria and/or bacterial fragments derived from bifidobacteria are obtained from less than 1×10¹¹ cfu bifidobacteria per g, based on dry weight of the final composition, more preferably less than 1×10¹⁰ cfu, even more preferably less than 1×10⁹ cfu. These numbers can be calculated by determining the amount of bifidobacteria in the mixture after incubation as in step a) and before step b), and subsequently taking into account how many gram of the present preparation is present in the final composition based on dry weight.

Additional ingredients that may be beneficial for obtaining the desired final nutritional composition may be added after process step a) or b). Preferably these are added after step a).

For an infant milk formula, ingredients such as skimmed milk, whey, lactose, vegetable fat, minerals, vitamins, as known in the art may be added.

Preferably the process comprises the additional steps of
d: incubating a substrate with Streptococcus thermophilus, preferably strain S. thermophilus CNCM 1-1620 or strain CNCM 1-1470, wherein the substrate is selected from the group consisting of milk, milk protein, whey, whey protein, whey protein hydrolysate, casein, casein hydrolysate, and lactose, to obtain an incubated mixture and
e: inactivating the S. thermophilus by heating the incubated mixture of step e and/or removing S. thermophilus cells from the incubated mixture of step e by centrifugation and/or filtration. Step e and b are preferably performed simultaneously. Preferably intact cells of streptococci are removed from the incubated product by physical elimination such as filtration and/or centrifugation, for example centrifugation for 1 h at 3000 g, with the intact cells remaining in the pellet or retentate and the product obtained by incubating a milk and/or milk-derived substrate with streptococci and subsequently inactivating the streptococcal cell fragments remaining in the supernatant and/or filtrate, respectively.

The heat inactivation and/or physical removal of living cells is such that the amount of living streptococci after treatment is below the detection limit as used by conventional plating techniques known in the art. This detection limit is less than 10⁴ cfu living cells of streptococci based on g dry weight composition, more preferably less than 10³ cfu/g. Hence, preferably in one embodiment according to the invention the preparation after step e) comprises less than 10³ cfu living streptococci per g dry weight of the preparation. Preferably the heat inactivation and/or removal step is such that at least 90, more preferably at least 99% of the cells present in the incubated mixture after step d) is eliminated.

Another preferred method for preparing the incubated product with S. thermophilus strains of the present invention is disclosed in EP 0778885, more particular in example 5 and 6. Another preferred method for preparing the incubated product with S. thermophilus strains of the present invention is disclosed in FR2723960 examples 2 to 6.

Preferably, an aqueous substrate comprising whey, whey protein and/or whey protein hydrolysate, is pasteurized, cooled and incubated with one or more Bifidobacterium strains, preferably B. breve strain CNCM 1-2219, upon which the incubated product is heat treated and stored. Preferably a second aqueous substrate comprising whey and/or lactose is incubated with S. thermophilus, preferably strain CNCM 1-1620 or strain CNCM 1-1470. Subsequently, the two incubated products are preferably mixed together and mixed with other components making up the nutritional composition. A fat component may or may not be included, but preferably a fat component is not yet included at this stage. Preferably, the mixture is preheated, and subsequently fat (also the term ‘lipids’ is used herein) is added in-line, homogenized, heat-treated and dried.

The incubation step d may be performed simultaneously with the incubation step with bifidobacteria in step a. Preferably the incubation with S. thermophilus is performed in a separate process step from the incubation with bifidobacteria. Separate incubation allows optimum conditions for each of the different bacteria and/or prevents unwanted interference of the different bacteria with the release of immunostimulatory components. Preferably the incubated mixture obtained after incubation of the substrate with streptococci is added to the mixture obtained in step a, step b, or step c, more preferably after step a). An improved effect on delayed hyper hypersensitivity response is observed when the present composition also comprised a mixture obtained after incubation with S. thermophilus. Thus in one embodiment the process according to the invention comprises the further step of:
f: combining the incubated mixture obtained in step d or e, preferably step d, with the incubated mixture obtained in step a, b, or c, preferably in step a. In one embodiment the incubated mixture obtained in step d is combined with the incubated mixture obtained in step a and steps b and e are performed simultaneously.

Preferably the final nutritional composition comprises from 2 to 94.5% based on dry weight of the preparation obtained by step d, more preferably from 5 to 80 wt. %, even more preferably from 5 to 40 wt. %. Preferably, the final nutritional composition comprises from 0.2 to 20 wt. % of a product obtained by step d per 100 ml, more preferably 0.5 to 14 wt. %, more preferably 1 to 10 wt. %, even more preferably 1 to 5 wt. % per 100 ml.

### Non-Digestible Carbohydrates

The preparation obtained by the present process comprises at least two different non-digestible carbohydrates. These non-digestible carbohydrates are added in process step c). The non-digestible carbohydrates advantageously stimulate the immune system. This stimulation may occur via an improvement of the intestinal microbiota and/or via a direct effect on the immune system. The presence of two different non-digestible carbohydrates synergistically improves the intestinal flora and/or synergistically stimulates the immune system.

The presence of both two non-digestible carbohydrates and a product obtained by incubating an aqueous substrate with bifidobacteria and subsequently inactivating and/or removing the bifidobacteria acts s increases resistance against infections to a larger extent. The improved effect between these two compounds was unexpected and cannot be explained by a symbiotic effect, wherein the non-digestible carbohydrates are specifically stimulating the growth of the beneficial micro-organisms present in the same preparation, since no living bifidobacteria are present in the incubated milk and/or milk-derived product.

The term “oligosaccharide” as used in the present invention refers to carbohydrates with a degree of polymerization (DP) of 2 to 250, preferably a DP 2 to 100, more preferably 2 to 60, even more preferably 2 to 10. If the oligosaccharide with a DP of 2 to 100 is included in the present preparation, this includes compositions which contain oligosaccharides with a DP between 2 and 5, a DP between 50 and 70 and a DP of 7 to 60. The term “non-digestible carbohydrate” as used in the present invention refers to carbohydrates which are not digested in the intestine by the action of acids or digestive enzymes present in the human upper digestive tract (small intestine and stomach) but which are preferably fermented by the human intestinal microbiota. For example, sucrose, lactose, maltose and maltodextrins are considered digestible.

Preferably the present non-digestible carbohydrate is soluble. The term “soluble” as used herein, when having reference to a carbohydrate, means that the carbohydrate is soluble according to the method described by L. Prosky et al., J. Assoc. Off. Anal. Chem. 71, 1017-1023 (1988).

Different non-digestible carbohydrates in the present invention relates to non-digestible carbohydrates differing in monosaccharide unit composition, or differing in degree of polymerization (DP) or both. Two non-digestible carbohydrates differ in monosaccharide composition when there is at least 30 mol % difference, more preferably at least 50 mol % difference in monosaccharide composition based on total mol monosaccharide units. For instance galacto-oligosaccharides with an average composition of Glu-Gal₃ and fructo-oligosaccharides with an average composition of Glu-Fru₃ differ for 75 mol %. Two non-digestible carbohydrates differ in DP if the average DP of the two carbohydrates differs more than 5 monosaccharide units, preferably more than 10 units, even more preferably more than 15 units. For example hydrolysed inulin with an average DP of 4 and long chain inulin with an average DP of 25 have a difference in DP of 21 units.

The non-digestible carbohydrates are at least one, more preferably at least two, selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides. Preferably the present preparation comprises fructo-oligosaccharides, galacto-oligosaccharides and/or galacturonic acid oligosaccharides, more preferably galacto-oligosaccharides, most preferably beta-galacto-oligosaccharides. The group of fructo-oligosaccharides includes inulin, the group of galacto-oligosaccharides includes transgalacto-oligosaccharides or beta-galacto-oligosaccharides, the group of gluco-oligosaccharides includes gentio-, nigero- and cyclodextrin-oligosaccharides and polydextrose, the group of arabinogalacto-oligosaccharides includes gum acacia, and the group of galactomanno-oligosaccharides includes partially hydrolysed guar gum.

For further improvement, the present non-digestible carbohydrates preferably have a relatively high content of short chain oligosaccharides, as these strongly stimulate the growth of bifidobacteria. Hence, preferably at least 10 wt. % of the non-digestible carbohydrates in the present preparation has a DP of 2 to 5 (i.e. 2, 3, 4, and/or 5) and at least 5 wt. % has a DP of 10 to 60. Preferably at least 50 wt. %, more preferably at least 75 wt. % of the non-digestible carbohydrates has a DP of 2 to 9 (i.e. 2, 3, 4, 5, 6, 7, 8, and/or 9).

More preferably the preparation obtained by the present process comprises galacto-oligosaccharides. The galacto-oligosaccharides are preferably selected from the group consisting of beta-galacto-oligosaccharides, lacto-N-tetraose (LNT), lacto-N-neotetraose (neo-LNT), fucosyl-lactose, fucosylated LNT and fucosylated neo-LNT. In a particularly preferred embodiment the present preparation comprises beta-galacto-oligosaccharides. Beta-galacto-oligosaccharides as used in the present invention refers to oligosaccharides composed of over 50%, preferably over 65% galactose units based on monomeric subunits, with a degree of polymerization (DP) of 2 to 20, in which at least 50%, more preferably at least 75%, even more preferably at least 90%, of the galactose units are linked together via a beta-glycosidic linkage, preferably a beta-1,4 glycosidic linkage. Beta-linkages are also predominant in human milk oligosaccharides. The average DP is preferably in the range of 3 to 6. A glucose unit may be present at the reducing end of the chain of galactose units. Beta-galacto-oligosaccharides are sometimes also referred to as transgalacto-oligosaccharides (TOS). A suitable source of beta-galacto-oligosaccharides is Vivinal®GOS (commercially available from Borculo Domo Ingredients, Zwolle, Netherlands). Other suitable sources are Oligomate (Yakult), Cupoligo, (Nissin) and Bi2muno (Classado). Beta-galacto-oligosaccharides were found to stimulate the growth of lactic acid producing bacteria, especially bifidobacteria.

Preferably the preparation obtained by the present process comprises fructo-oligosaccharides. Fructo-oligosaccharides as used in the present invention refers to carbohydrates composed of over 50%, preferably over 65% fructose units based on monomeric subunits, in which at least 50%, more preferably at least 75%, even more preferably at least 90%, of the fructose units are linked together via a beta-glycosidic linkage, preferably a beta-2,1 glycosidic linkage. A glucose unit may be present at the reducing end of the chain of fructose units. Preferably the fructo-oligosaccharide has a DP or average DP in the range of 2 to 250, more preferably 2 to 100, even more preferably 10 to 60. Fructo-oligosaccaride comprises levan, hydrolysed levan, inulin, hydrolysed inulin, and synthesised fructo-oligosaccharides.

Preferably the preparation comprises short chain fructo-oligosaccharides with an average degree of polymerization (DP) of 3 to 6, more preferably hydrolysed inulin or synthetic fructo-oligosaccharide. Preferably the preparation comprises long chain fructo-oligosaccharides with an average DP above 20. Preferably the preparation comprises both short chain and long chain fructo-oligosaccharides. Fructo-oligosaccharide suitable for use in the process of the invention is also readily commercially available, e.g. RaftilineHP (Orafti).

More preferably the preparation obtained by the process according to the invention comprises a combination of galacto-oligosaccharides and fructo-oligosaccharides, more preferably long chain fructo-oligosaccharides. Such a mixture synergistically stimulates the growth of a healthy intestinal microbiota, particularly bifidobacteria.

The preparation obtained by the process according to the invention preferably comprises uronic acid oligosaccharides, more preferably mannonuric acid and/or galacturonic acid oligosaccharides, even more preferably galacturonic acid. The term uronic acid oligosaccharide as used in the present invention refers to an oligosaccharide wherein at least 50% of the monosaccharide units present in the oligosaccharide is uronic acid. The term galacturonic acid oligosaccharide as used in the present invention refers to an oligosaccharide wherein at least 50% of the monosaccharide units present in the oligosaccharide is galacturonic acid. The galacturonic acid oligosaccharides used in the invention are preferably prepared from degradation of pectin, pectate, and/or polygalacturonic acid Preferably the degraded pectin is prepared by hydrolysis and/or beta-elimination of fruit and/or vegetable pectins, more preferably apple, citrus and/or sugar beet pectin, even more preferably apple, citrus and/or sugar beet pectin degraded by at least one lyase. In a preferred embodiment, at least one of the terminal galacturonic acid units of the galacturonic acid oligosaccharide has a double bond. The double bond effectively protects against attachment of pathogenic bacteria to intestinal epithelial cells. Preferably one of the terminal galacturonic acid units comprises a C₄-C₅ double bond. The galacturonic acid oligosaccharide can be derivatised. The galacturonic acid oligosaccharide may be methoxylated and/or amidated. Preferably the galacturonic acid oligosaccharides are characterised by a degree of methoxylation above 20%, preferably above 50% even more preferably above 70%. Uronic acid oligosaccharides advantageously reduce the adhesion of pathogenic micro-organisms to the intestinal epithelial cells. Furthermore, uronic acid oligosaccharides stimulate the immune system by increasing the Th1 response.

Thus, in one embodiment the preparation obtained by the process according to the invention and for use according to the present invention preferably comprises at least beta-galacto-oligosaccharides. In one embodiment the preparation obtained by the process according to the invention and for use according to the present invention preferably comprises at least short chain fructo-oligosaccharides and/or long chain fructo-oligosaccharides, preferably long chain fructo-oligosaccharides. In one embodiment the preparation obtained by the process according to the invention and for use according to the present invention preferably comprises at least uronic acid oligosaccharides. In one embodiment the preparation for use according to the present invention preferably comprises at least beta-galacto-oligosaccharides and at least short chain fructo-oligosaccharides or long chain fructo-oligosaccharides or both. In one embodiment the preparation for use according to the present invention preferably comprises at least beta-galacto-oligosaccharides and at least uronic acid oligosaccharides. In one embodiment the preparation for use according to the present invention preferably comprises at least short chain fructo-oligosaccharides and uronic acid oligosaccharides or long chain fructo-oligosaccharides and uronic acid oligosaccharides. In one embodiment the preparation for use according to the present invention preferably comprises at least beta-galacto-oligosaccharides and short chain fructo-oligosaccharides and uronic acid oligosaccharides or at least beta-galacto-oligosaccharides and long chain fructo-oligosaccharides and uronic acid oligosaccharides.

Preferably the weight ratio between the mixture of two different non-digestible carbohydrates, preferably beta-galacto-oligosaccharides and fructo-oligosaccharide, is between 20 and 0.05, more preferably between 20 and 1. Beta-galacto-oligosaccharides are more reminiscent to the human milk oligosaccharides. Preferably the present preparation comprises beta-galacto-oligosaccharides with a DP of 2-10 and/or fructo-oligosaccharides with a DP of 2-60. This combination was found to synergistically increase bifidobacteria and lactobacilli. The presence of these three non-digestible oligosaccharides even further stimulates the bifidobacteria. The weight ratio transgalacto-oligosaccharide: fructo-oligosaccharide: uronic acid oligosaccharide is preferably (20 to 2):1:(1 to 20), more preferably (12 to 7):1:(1 to 3).

Preferably, the final nutritional composition consisting of or comprising the preparation obtained by the process according to the invention comprises 80 mg to 2 g non-digestible carbohydrates per 100 ml, more preferably 150 mg to 1.50 g, even more preferably 300 mg to 1 g. Based on dry weight, the nutritional composition preferably comprises 0.25 wt. % to 20 wt. %, more preferably 0.5 wt. % to 10 wt. %, even more preferably 1.5 wt. % to 7.5 wt. % non-digestible carbohydrates. A lower amount of non-digestible carbohydrate will be less effective in stimulating the immune system and/or beneficial bacteria in the microbiota, whereas a too high amount will result in side-effects of bloating and abdominal discomfort.

The two different non-digestible carbohydrates are added (i.e. step c) after step a) preferably immediately prior to step b) or after step b), preferably step c) is conducted after step b), preferably step c) is conducted after step e), i.e. after inactivation by heat treatment and/or removal of the bifidobacteria and optionally S. thermophilus.

Preferably the preparation obtained by the process according to the invention comprises an aqueous substrate comprising at least one of the group selected from milk, milk protein, whey, whey protein, whey protein hydrolysate and lactose incubated with B. breve, more preferably strain CNCM 1-2219, and at least one, preferably two non-digestible carbohydrates from the group consisting of galacto-oligosaccharides and fructo-oligosaccharides.

In one aspect the present invention concerns the present process, wherein in step c only one non-digestible carbohydrate is added. It is particularly advantageous that the only one non digestible carbohydrate is fructo-oligosaccharide.

In one aspect, the invention concerns a preparation obtainable by the process according to the present invention as described above. In one embodiment the final nutritional composition consisting of or comprising the preparation obtained by the present process comprises 0.5 to 10 g non-digestible carbohydrate as defined above per 100 g dry weight of the composition. In one embodiment the final nutritional composition consisting of or comprising the preparation obtained by the present process has a viscosity of 1 to 60 mPa.s at a shear rate of 100 s⁻¹ at 20° C.

Preferably the above process comprises a drying step. Drying steps are known in the art. A suitable drying step is spray drying. Preferably the drying step is performed in such a way that the dried product is a powder comprising less than 10 wt. % water, more preferably less than 5 wt. %. Preferably the drying step is performed after step c. Alternatively, the drying step may be performed after step b and/or after step e, after which the non-digestible oligosaccharides are dry blended in the product.

### Nutrition

It was found that the present preparation can be advantageously applied in food, such as baby food and clinical food. The present preparation or composition comprising the present preparation is preferably enterally administered, more preferably orally. Preferably the composition is a complete nutrition.

Preferably the nutrition is suitable for administration to infants. More preferably the present nutritional composition is an infant or follow on formula. The present composition can be advantageously applied as a complete nutrition for infants.

Preferably the present composition is an infant nutrition comprising based on dry weight of the infant nutrition
i) from 0.5 to 10 wt. % of the sum of galacto-oligosaccharides and fructo-oligosaccharides, and
ii) from 5 to 99.5 wt. % of the preparation obtained after step b according to the present process, wherein the bifidobacteria in step a belong to the species B. breve, preferably strain B. breve CNCM 1-2219
iii) and optionally 2 to 94.5 wt. % of the preparation obtained after step e.

Such nutrition preferably comprises lipid, protein and carbohydrate and is preferably administered in liquid form. The term “liquid food” as used in the present invention includes dry food (e.g. powders) which are accompanied with instructions as to admix said dry food mixture with a suitable liquid (e.g. water).

Hence, the nutritional composition of the present invention preferably comprises between 5 and 60% lipids based on total of calories, between 5 and 60% protein based on total calories, between 15 and 90% digestible carbohydrate based on total calories. Preferably the present nutritional composition comprises between 5 and 30% lipid based on total calories, between 15 and 40% protein based on total calories and between 25 and 75% digestible carbohydrate based on total calories when intended for adult human subjects. Preferably the present nutritional composition comprises between 30 and 60% lipid based on total calories, between 5 and 15% protein based on total calories and between 25 and 75% digestible carbohydrate based on total calories, more preferably 35 to 50% lipids based on total calories, 7.5 to 12.5% proteins based on total calories, and 40 to 55% digestible carbohydrate based on total calories when intended for infants. For calculation of the % protein based on total calories, the total of calories provided by proteins, peptides and amino acids needs to be taken into account.

Preferably the lipids comprise vegetable oils. The vegetable lipid is preferably at least one selected from the group consisting of soy oil, palm oil, coconut oil, safflower oil, sunflower oil, corn oil, canola oil and lecithins. Preferably a combination of vegetable lipids and at least one oil selected from the group consisting of fish oil and omega-3 containing vegetable, algae or bacterial oil is used. In a preferred embodiment, the present method further comprises the administration of long-chain polyunsaturated acid (LC-PUFA). As it is believed that these act on the immune system via a mechanism different from the non-digestible carbohydrates and the product obtained by incubating a milk and/or milk-derived substrate with bifidobacteria and subsequently inactivating the bifidobacteria, the combination of the present invention with the LC-PUFA is deemed to act synergistically.

The nutritional composition of the present invention preferably comprises between 5 and 60% lipids based on total of calories, preferably between 5 and 30% lipid based on total calories when intended for adults, preferably between 30 and 60% lipid based on total calories, more preferably 35 to 50% lipids based on total calories, when intended for infants.

The proteins used in the nutritional preparation are preferably selected from the group of non-human animal proteins (such as milk proteins, meat proteins and egg proteins), vegetable proteins (such as soy protein, wheat protein, rice protein, and pea protein), hydrolysates thereof, free amino acids and mixtures of proteins, hydrolysates and free amino acids. Cow milk proteins such as casein and whey proteins are particularly preferred. As the present composition is suitably used to reduce the allergic reactions, especially in infants, the protein of is preferably selected from the group consisting of hydrolyzed milk protein. Preferably the present composition comprises hydrolyzed casein and/or hydrolyzed whey protein, hydrolyzed vegetable protein and/or free amino acids, most preferably hydrolyzed whey protein. The use of these proteins further reduced the allergic reactions. The use of these hydrolysed proteins advantageously improves the absorption of the dietary protein component. This is especially advantageous for infants and for subjects suffering from a disorder.

The nutritional composition of the present invention preferably comprises between 5 and 60% protein based on total calories, preferably between 15 and 40% protein based on total calories when intended for adult human subjects, and preferably between 5 and 15% protein based on total calories and more preferably 7.5 to 12.5% proteins based on total calories, calories when intended for infants. For calculation of the % protein based on total calories, the total of calories provided by proteins, peptides and amino acids needs to be taken into account.

A source of digestible carbohydrate may be added to the nutritional formula. Any suitable (source of) digestible carbohydrate may be used, for example sucrose, lactose, glucose, fructose, corn syrup solids, and maltodextrins, and mixtures thereof. Hence, the nutritional composition of the present invention preferably comprises between 15 and 90% carbohydrate based on total calories, more preferable between 25 and 75% carbohydrate based on total calories, more between 40 to 55% carbohydrate based on total calories.

The nutritional composition of the present invention is preferably is in liquid form. It preferably has a limited viscosity. It was found that the present process provides a liquid nutrition with sufficiently low viscosity so it can be applied as e.g. liquid baby foods and liquid clinical food which can be fed through a teat, a tube or a straw, while retaining the low viscosity. In a preferred embodiment, the present composition has a viscosity below 600 mPa.s, preferably below 250 mPas, more preferably below 60 mPa.s, even more preferably below 35 mPa.s, most preferably below 6 mPa.s, at a shear rate of 100 s⁻¹ at 20 ° C. Whenever the term viscosity used in the present document, this refers to the physical parameter which is determined according to the following method: The viscosity may be determined using a Carri-Med CSL rheometer. The used geometry is of conical shape (6 cm 2 deg acrylic cone) and the gap between plate and geometry is set on 55 um. A linear continuous ramp shear rate is used from 0 to 150 s⁻¹ in 20 seconds. It is noted that a composition in powder form with the instruction to prepare an aqueous solution, e.g. by adding water in a certain ratio and which then results in a viscosity as specified is also encompassed by the invention.

Stool irregularities (e. g. hard stools, insufficient stool volume, diarrhoea) is a major problem in many babies and ill subjects that receive liquid foods. It was found that stool problems may be reduced by administering the present preparation in liquid food which has an osmolality between 50 and 500 mOsm/kg, more preferably between 100 and 400 mOsm/kg.

In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml. When used as an infant formula the caloric density is most preferably between 0.6 and 0.8 kcal/ml.

### Application

The present preparation obtained by the present process was found to synergistically decrease bacterial translocation and/or decrease intestinal permeability. The present preparation can advantageously be used in the treatment and/or prevention of a disease, and thus the invention concerns a method for the treatment and/or prevention of a disease in a mammal, said method comprising administering the present preparation to the mammal In other words, the invention also concerns the use of a preparation according to the present invention for the manufacture of a composition, preferably a nutritional composition, for the treatment and/or prevention of a disease. In other words the invention concerns a preparation or nutritional composition comprising a preparation according to the present invention for use in the treatment and/or prevention of a disease. Preferably the mammal is a human, even more preferably a human infant. Thus the invention also concerns the use of a preparation according to the present invention for the manufacture of a composition, preferably a nutritional preparation, for the treatment and/or prevention of a disease in an infant. Or in other words the invention concerns a preparation or nutritional composition comprising a preparation according to the present invention for use in the treatment and/or prevention of a disease in an infant.

In the context of this invention, an infant is in the age of 0 to 6 years, preferably in the age of 0 to 4 years, preferably in the age of 0 to 2 years, preferably in the age of 0 to 1 year.

Also the invention concerns a method for providing nutrition to an infant, said method comprising administering the present preparation or nutritional composition to the infant. In other words, the invention also concerns the use of a preparation according to the present invention for the manufacture of a nutritional composition for providing nutrition to an infant. In other words the invention concerns a preparation or nutritional composition comprising a preparation according to the present invention for use in providing nutrition to an infant.

It was found that the present preparation decreases bacterial translocation from the intestine across the intestinal barrier. This translocation may occur paracellular, for instance via the tight junctions, or may occur transcellular, such as the bacterial translocation observed with (opportunistic) invasive bacteria. The present preparation can hence advantageously be used for treatment and/or prevention of bacterial translocation, in particular of invasive and opportunistic invasive bacteria such as E. coli, species belonging to the genus Salmonella, Shigella, Campylobacter, Yersinia, and Listeria Pseudomonas aeruginosa, Klebsiella, Proteus, Enterobacter, Citrobacter, more preferably Pseudomonas and Salmonella.

Opportunistic invasive bacteria are bacteria that are not invasive in normal, healthy subjects, but which become invasive in a subject which has a suppressed or compromised immune system or a compromised intestinal barrier. Examples of subjects with a compromised immune system are neutropenic oncology patients, patients suffering from cystic fibrosis, AIDS patients, and HIV infected patients. Examples of subjects with a compromised intestinal barrier are patients suffering from burn wounds, patients suffering from haemorrhagic shock and critically ill patients.

Suitable model systems to study the effect of the preparation on intestinal barrier function and/or maturation include transwell monolayers of intestinal epithelial cells, Ussing chambers using cultured intestinal epithelial cells or intestinal epithelial tissues and animal models. In such models the transwell electrical transepithelial electrical resistance or translocation of molecules and/or bacteria can be determined A suitable animal model is disclosed in example 1. Another suitable animal model is disclosed in Koh et al, 2005, Infection & Immunity 73: 2262-2272.

The present preparation is suitable for the treatment and/or prevention of infections. The present preparation can be advantageously used for the treatment and/or prevention of intestinal infections, systemic infections and/or respiratory tract infections, in particular infections caused by invasive bacteria or opportunistic invasive bacteria. The present preparation is suitable for the treatment and/or prevention of diarrhoea, in particular diarrhoea caused by invasive bacteria or opportunistic invasive bacteria.

In the context of the present invention, ‘prevention’ of a disease or cetain disorder also means ‘treatment of a person at risk’ of a disease or certain disorder.

It was found that the present preparation decreases bacterial translocation from the intestine across the intestinal barrier. Hence, the present preparation is suitable for improvement of the intestinal barrier function, for increasing intestinal barrier maturation and/or decreasing intestinal barrier permeability. Thus in one embodiment the invention concerns the use of a preparation according to the present invention for the manufacture of a composition for use in improving intestinal barrier function, increasing intestinal barrier maturation and/or decreasing intestinal barrier permeability. In other words the invention concerns a preparation according to the present invention for use in improving intestinal barrier function, increasing intestinal barrier maturation and/or decreasing intestinal barrier permeability. The improvement of the intestinal barrier function, increasing intestinal barrier maturation and/or decreasing intestinal barrier permeability preferably changes towards levels observed in healthy breast fed infants.

### EXAMPLE 1

### Enhanced Effect of Milk-Derived Product Incubated by Lactic Acid Producing Bacteria and Non-Digestible Carbohydrates on Infection

Male BALB/c mice, n=25 per group and 3-4 weeks of age, received the following diets for 4 weeks at lib.
1. An AIN-93 (balanced semi-synthetic rodent chow) mixed 1:1 (w/w) with the infant milk formula Gallia Apaisia 1. Apaisia 1 is a commercially available IMF comprising an incubated aqueous substrate (comprising milk, milk protein, whey, whey protein, and/or lactose), obtained by separate incubation with Streptococcus thermophilus CNCM-1620 and Bifidobacterium breve CNCM 1-2219. In this preparation S. thermophilus and B. breve incubated mixtures obtained in step a) and d) are heat killed immediately after the incubation step (step b) and e), respectively. The final amount of the with B. breve incubated aqueous substrate mixture is about 16 wt. % based on dry weight of the IMF.

2. An AIN-93 (balanced semi-synthetic rodent chow) mixed 1:1 (w/w) with the infant milk formula Gallia Apaisia 1 to which 2 wt. % based on dry weight of a mixture of non-digestible carbohydrates (GF) was added containing trans-galacto-oligosaccharides (source: Vivinal GOS) and inulin (Raftilin HP) in a w/w/ratio of 9/1 was added, (step d). The final concentration of GF in the mouse diet was 1 wt.%.

3.An AIN-93 (balanced semi-synthetic rodent chow) to which fat and lactose was added in order to make the fat and lactose content the same as in diet 1 and 2.

Table 1 shows the composition of the tested diets. The amount of carbohydrates, lactose, fats, proteins and fibers were comparable for all diets.

TABLE 1
Composition of the tested diets

| diet | TOS:Inulin 9:1 w/w | Bioconverted product |
|---|---|---|
| 1 | — | + |
| 2 | 1 wt. % | + |
| 3 | — | − |

Subsequently a Salmonella infection was introduced by Salmonella enteritidis LMG22715 oral gavage, 2×10⁵ cfu/mouse in 0.2 ml buffer. After 6 days of infection the mice were sacrificed and bacterial translocation to the organs was measured as one of the main parameters. During the infection period the diet regime was not changed. Also illness score was measured. The illness score is an indication of illness based on weight loss, reactivity and alertness, and fur condition.

### Results

Food intake and growth was comparable in all groups in the period before infection was introduced.

Table 2 shows the results on general health, and bacterial translocation (BT) to different organs.

Illness score and bacterial translocation to the liver and spleen was lower in group 2 than in group 1 due to the additional presence of GF. BT log to the liver, spleen, lung and kidney was lower in 2 than in 1 because of GF.

TABLE 2
Illness score and bacterial translocation (log
number of cfu per g tissue in log) on day 34.

| | Illness | BT log | BT log | BT log | BT log |
|---|---|---|---|---|---|
| Diet | score | liver | spleen | kidney | lung |
| 1 | 4.85 | 3.80 | 3.70 | 2.56 | 2.75 |
| 2 | 3.65 | 2.37 | 2.40 | 1.89 | 2.05 |
| 3 | 4.93 | 3.85 | 4.20 | 3.15 | 3.20 |

The results of this experiment are an indication that the present invention can advantageously be used for treatment and/or prevention of infections, and/or bacterial translocation and/or for improvement of the intestinal barrier function.

### Example 2

### Infant Milk Formula

Pasteurized skimmed cow's milk was concentrated by evaporation to about 43 wt. % dry matter based on weight of the skimmed cow's milk. The concentrate was cooled to about 37° C., was then inoculated with about 10% (v/w) B. breve CNCM 1-2219 culture comprising 3×10⁹ cfu per ml. This inoculum was prepared as known in the art. The initial pH was between 6-7.1. After incubating for 8 h at 37° C., in a tank with periodic stirring for 10 minutes every 2 hours, the pH stayed between 6-7.1 and the B. breve population was about 10⁶ bacteria/ml (step (a)).

A pre-warmed inoculum was prepared from S. thermophilus CNCM 1-1620 culture by maintaining a frozen inoculum for about 7 h at about 40 ° C. Pasteurized lactose solution (between 350 and 450 g/l) was cooled to about 45 and 55 ° C., and then inoculated with about 10% (v/w) S. thermophilus CNCM 1-1620 pre-warmed inoculum comprising about 3×10⁹ cfu per ml. The initial pH was about pH 6. After incubating for about 7 h at about 50° C., in a tank with periodic stirring for 10 minutes every 2 hours, the pH was kept constant between 6-8 and the S. thermophilus population was about 10⁶ bacteria/ml (step (d)).

Both incubated preparations, skim milk, vegetable fat, malto-dextrin, trans-galacto-oligosaccharides and fructo-oligosaccharides oligosaccharides, and other ingredients well known for infant milk formula (such as vitamins, minerals, trace elements) were mixed (step c, f).

The mixture was pasteurized (step b, e) and subsequently spray-dried.

Final composition of the infant formula comprising per 100 ml:
68 kcal
1.45 g protein (casein and whey protein from milk; partially hydrolysed)
8.6 g digestible carbohydrates (mainly lactose and maltodextrin)
3.1 g fats (mainly vegetable fats)
0.8 g trans-galactoligosaccharides (source VivinalGOS) and polyfructose (source raftilinHP)
Trace elements, minerals, vitamins and other micronutrients (taurine, choline, inositol, nucleotides, carnitine) as known in the art.

1. A process for the manufacture of a preparation comprising the steps of:
(a) incubating an aqueous substrate with bifidobacteria, wherein the substrate comprises at least one selected from the group consisting of milk, milk protein, whey, whey protein, whey protein hydrolysate, casein hydrolysate, and lactose to obtain an incubated mixture; and
(b) combining the incubated mixture with at least two different non-digestible carbohydrates, wherein at least one of the non-digestible carbohydrates is selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, raffinose, lactosucrose, sialic acid comprising oligosaccharides and uronic acid oligosaccharides.

2. The process of claim 1, further comprising inactivating the bifidobacteria.

3. The process of claim 2, in which the bifidobacteria is inactivated by heating the incubated mixture and/or removing bifidobacteria cells from the incubated mixture by centrifugation and/or filtration.

4. The process according to claim 2, wherein the preparation comprises less than 10³ cfu living bifidobacteria per gram dry weight of the preparation.

5. The process according to claim 1, in which both of the at least two different non-digestible carbohydrates are selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, raffinose, lactosucrose, sialic acid comprising oligosaccharides and uronic acid oligosaccharides.

6. The process according to claim 1, wherein at least one, optionally both, of the at least two different non-digestible carbohydrates is selected from the group consisting of galacto-oligosaccharides and fructo-oligosaccharides.

7. The process according to claim 1, in which the bifidobacteria comprises the species B. breve.

8. The process according to claim 7, in which the species B. breve is strain B. breve CNCM 1-2219.

9. The process according to claim 1, further comprising at (c) combining the incubated mixture with a second incubated mixture prepared as follows:
(i) incubating a substrate with Streptococcus thermophilus, wherein the substrate is selected from the group consisting of milk, milk protein, whey, whey protein, whey protein hydrolysate, casein, casein hydrolysate, and lactose, to obtain an incubated mixture; and, optionally,
(ii) inactivating the S. thermophilus by heating the incubated mixture of step d and/or removing S. thermophilus cells from the incubated mixture of step e by centrifugation and/or filtration.

10. The process according to claim 9, in which the Streptococcus thermophilus comprises strain S. thermophilus CNCM 1-1620, CNCM 1-1470, or both.

11. The process according to claim 1, further comprising (c) drying the preparation.

12. The process according to claim 1, in which at least one of the non-digestible carbohydrates is fructo-oligosaccharide.

13. A preparation obtained by the process according to claim 1.

14. The preparation according to claim 13 comprising 0.5 to 10 grams of non-digestible carbohydrate per 100 g dry weight of the composition.

15. An infant nutritional composition comprising, based on dry weight of the composition,
(a) 0.5 to 10 wt. % the sum of galacto-oligosaccharides and fructo-oligosaccharides, and
(b) from 5 to 99.5 wt. % of the preparation obtained from claim 1, wherein the bifidobacteria belongs to the species B. breve.

16. The composition of claim 15, further comprising (c) 2 to 94.5 wt. % of a preparation obtainable by:
(a) incubating an aqueous substrate with bifidobacteria, wherein the substrate comprises at least one selected from the group consisting of milk, milk protein, whey, whey protein, whey protein hydrolysate, casein hydrolysate, and lactose to obtain an incubated mixture; and
(b) combining the incubated mixture with at least two different non-digestible carbohydrates, wherein at least one of the non-digestible carbohydrates is selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, raffinose, lactosucrose, sialic acid comprising oligosaccharides and uronic acid oligosaccharides.

17. A method of providing nutrition to an infant, comprising administering to the infant the preparation according to claim 13.

18. The method according to claim 17, in which the infant suffers from infections and/or diarrhoea.

19. The method according to claim 18, in which the infection is bacterial translocation.

20. A method of improving intestinal barrier function, increasing intestinal barrier maturation and/or decreasing intestinal barrier permeability of a mammal, comprising administering to the mammal a composition according to claim 5.
